(19) United States
(12) Patent Application Publication
(10) Pub. No.: US 2025/0160305 A1
(43) Pub. Date: May 22, 2025

(54) ESTABLISHMENT AND APPLICATION OF MURINE MODELS OF HYPERURICEMIA-EXACERBATED PERIODONTITIS

(71) Applicant: HOSPITAL OF STOMATOLOGY, GUANGZHOU MEDICAL UNIVERSITY (YANGCHENG HOSPITAL OF, Guangzhou (CN)

(72) Inventors: Ting YU, Guangzhou (CN); Zhicong WU, Guangzhou (CN); Yi GUO, Guangzhou (CN); Xinhong WANG, Guangzhou (CN)

(21) Appl. No.: 19/027,085

(22) Filed: Jan. 17, 2025

Related U.S. Application Data
(63) Continuation of application No. PCT/CN2024/079709, filed on Mar. 1, 2024.

(30) Foreign Application Priority Data
Mar. 2, 2023 (CN) 202310190961.2

(51) Int. Cl. A01K 67/00; A61D 7/00

(52) U.S. Cl. CPC A01K 67/00; A61D 7/00; A01K 2207/20; A01K 2227/105; A01K 2267/03

(57) ABSTRACT

Disclosure is a composite mouse model for simulating the exacerbation of periodontitis by hyperuricemia, as well as the establishment methods and applications thereof. The establishment methods includes: intraperitoneally injecting potassium oxonate into a mouse continuously for 7 days to establish a hyperuricemia model; ligating the maxillary second molar of the mouse with a surgical silk thread to construct a periodontitis model; continuously intraperitoneally injecting potassium oxonate into the mouse for another 7 days (days 8 to 14) to establish a composite mouse model that simulates exacerbation of periodontitis by hyperuricemia; continuously intraperitoneally injecting potassium oxonate for 14 days (days 15 to 28) at a fixed frequency once a day to maintain stability of the hyperuricemia model and constructing a stable hyperuricemia compound periodontitis mouse model; and on the 15th day, intraperitoneally injected allopurinol for 14 days continuously to decrease uric acid.

### CROSS-REFERENCE TO RELATED APPLICATIONS

The present application is a continuation application of International Application No. PCT/CN2024/079709, filed on Mar. 1, 2024, which claims priority to Chinese Patent Application No. 202310190961.2, filed on Mar. 2, 2023. The entire contents of the above-mentioned applications are incorporated herein by reference.

### TECHNICAL FIELD

The present application relates to the technical field of biological experimental model construction, and in particular to a composite mouse model for simulating exacerbation of periodontitis by hyperuricemia, as well as establishment method and use thereof.

### BACKGROUND

Periodontitis is a chronic multifactorial inflammatory disease characterized by the destruction of periodontal tissues and caused by abnormal plaque biofilm. Specific microbiota cause progressive destruction of periodontal tissues by inducing dysregulated host inflammatory response, ultimately destroying connective tissues and alveolar bone. Severe periodontitis is the sixth most common disease in humans, which affects nearly 24% of global population. Severe periodontitis can not only lead to tooth loss which seriously impair quality of life but also have adverse effects on general health. Relevant studies have shown that the “three highs” including hyperglycemia, hypertension, and hyperlipidemia are major risk factors for periodontitis, and their relationships with periodontitis have been proven to varying degrees. Hyperuricemia, one of the metabolic syndromes, has become the “fourth high” after the “three highs”. Hyperuricemia is a common comorbidity and potential risk factor for cardiovascular disease, chronic kidney disease, and diabetes. Whether there is a relationship between hyperuricemia and periodontitis, and whether it is an intermediate factor linking periodontitis to diabetes, cardiovascular disease, and metabolic syndrome, has become a new public health issue.

Hyperuricemia is a metabolic disease caused by overproduction of urate and/or insufficient intestinal excretion and/or renal urate excretion, characterized by excessive elevation of blood uric acid concentration, and its prevalence has been increasing in recent years. According to reports, hyperuricemia affects about 15% to 20% of the world's population, and the number of hyperuricemia people in China is as high as 150 million. Recent studies suggest that hyperuricemia may be a novel risk indicator for periodontitis. A retrospective cohort study showed that patients with gout (an inflammatory disease caused by the deposition of sodium urate crystals in joints and periarticular tissues due to abnormally elevated blood uric acid levels) had a 13% higher risk of periodontitis than the healthy group (HR=1.13, 95% CI=1.10-1.16). Colchicine, a medication used to treat gout, effectively reduced the risk of developing periodontitis (HR=0.85, 95% CI=0.79-0.91). Animal studies found that febuxostat, a urate-lowering drug, reduced the progression of periodontitis in rats. A cross-sectional study found that increased serum uric acid was associated with localized stage II/III periodontitis (OR=1.10, 95% CI=1.00-1.21). The results of a previous systematic review study by our research group showed that compared with healthy controls, the blood uric acid concentration in patients with periodontitis was increased (WMI)=1.00 mg/dL, 95% CI=0.63-1.37, P<0.001). The salivary uric acid concentration was decreased (SMI)=−0.95, 95% CI=−1.23-−0.68, P<0.001). Non-surgical periodontal treatment has also been shown to reduce serum uric acid in periodontitis patients. Several studies do not support the association between hyperuricemia and periodontitis. A cross-sectional study (8809 hyperuricemia patients and 126465 controls) found no significant association between hyperuricemia and a diagnosis history of periodontitis (O) R=0.89, 95% CI=0.81-0.96). Another cross-sectional study conducted in the same country (12,735 Korean adults) found a significant positive association between periodontitis and hypouricemia (rather than hyperuricemia) (OR=1.62, 95% CI=1.13-2.33). In summary, there is little evidence of a direct link between hyperuricemia and periodontal disease. Relevant studies mainly focus on the relationship between periodontitis and changes in blood uric acid levels within the normal range, rather than on the relationship between periodontitis and hyperuricemia. The few studies that have directly focused on periodontitis and hyperuricemia have drawn seemingly contradictory conclusions. Moreover, there is still a lack of animal experimental evidence to establish a link between the two at the level of biological mechanisms. Therefore, the development of an animal model that can simulate the aggravation of periodontitis by hyperuricemia is very important to verify and replicate the clinical research results and explore the biological mechanisms between the two diseases.

Currently, there is only one report on the animal model of hyperuricemia exacerbating periodontitis, which comes from animal experiments of our research group. A mouse model of hyperuricemia combined with periodontitis was established by feeding a diet supplemented with 5% potassium oxonate and 2.5% uric acid for 35 days and ligating the maxillary second molar with silk thread on the 25th day. This study measured serum uric acid levels in mice at the end of the experiment and performed micro-CT analysis on the maxillary bones. The results showed that the blood uric acid levels in the potassium oxonate and uric acid-containing diet feeding groups increased, but morphological analyses in maxillary bones with micro-CT did not find any aggravating effect of hyperuricemia (or elevated uric acid levels) on the destruction of periodontitis. It must be pointed out that the mouse model of hyperuricemia combined with periodontitis established by this method through diet feeding and ligation methods did not find any correlation between uric acid concentration and alveolar bone resorption. This may be due to the following reasons: (1) although the periodontitis model was successfully constructed, the observation time was not long enough, and the potential adverse effects of hyperuricemia on alveolar bone resorption were not apparent in a short term; (2) the sample size was small, and errors caused by individual differences masked the possible adverse effects of hyperuricemia. Because of this, our research group then tried to re-establish a hyperuricemia model with a long term duration by feeding mice with a same diet containing potassium oxonate and uric acid for 35, 43 and 53 days. After blood was collected at the end of the experiment (days 35, 43 and 53), the serum uric acid level was detected by a fully automatic biochemical analyzer. The results showed that there was no significant difference in the serum uric acid level between test group and the normal feeding group. Therefore, the technique of establishing a hyperuricemia model by diet feeding is not stable. This also partly explains why the “hyperuricemia” mouse model established earlier by our research group failed to aggravate the bone destruction of periodontitis in mice. To sum up, there is no report of an animal model successfully simulating hyperuricemia aggravating periodontitis currently; the new animal model of hyperuricemia-exacerbated periodontitis is urgently needed to fill the gap in this research field.

At present, the methods for constructing an animal model of periodontitis or hyperuricemia are relatively mature and diverse. However, in the technical field of constructing mouse models of hyperuricemia combined with periodontitis, there are very few reports globally, and there has been no previous report on the successful construction of a mouse model in which hyperuricemia aggravates periodontitis. To achieve this goal, the main difficulties are as follows. Firstly, similar to the pathological process of periodontitis in humans in which bacterial plaque is the initiating factor, ligation can lead to local plaque accumulation and induce the formation of periodontitis. Therefore, ligation is currently the most typical method to construct animal periodontitis models. However, a careful review of the literature revealed that although mice represent the most convenient, cheapest, and most versatile model animal, ligation method is more commonly used in rats and other bigger animals for induction of periodontitis while mice are favored in oral gavage method. The current ligation operation may cause great risk of damage on the gums of animals, and sharp operating instruments can easily cause oral bleeding in mice. In severe cases, blood flows into the respiratory tract, leading to the death of mice. A small space of oral cavity of mice also brings severe technical challenges to ligation. Secondly, due to the presence of uricase, the uric acid level cannot be stably maintained at a high level in the hyperuricemia model constructed by supplementing exogenous uric acid or uric acid precursors or by inhibiting renal uric acid excretion. Currently, potassium oxonate is the most commonly used chemical to construct hyperuricemia models. It can competitively bind to uricase, inhibit uricase activity, and increase blood uric acid levels in a short time and maintain it for a long time. This method can establish a hyperuricemia model in a short time (two weeks). However, the maintenance of the hyperuricemia model still requires a long-term administration. If the dosing concentration and frequency are too low, the uric acid levels cannot be maintained stably. If the dosing concentration and frequency are too high, it is easy to cause damage and strong inflammatory reactions in kidney and other adverse effects in experimental animals. Therefore, in medium- and long-term experiments, it is particularly important to explore an appropriate dosing concentration and frequency to maintain a higher level of uric acid without causing evident adverse effects such as strong inflammatory response in kidney. Thirdly, establishment of combined hyperuricemia and periodontitis models is not merely a simple superposition of a hyperuricemia model and a periodontitis model. When exposure factors and research purposes are different (for example, when studying the impact of hyperuricemia on periodontitis, the former is an exposure factor; conversely, periodontitis is an exposure factor; when studying the relationship or joint interaction between the two diseases, they are exposure factors to each other), the duration of ligation and administration for hyperuricemia are also different. The key issue is that the impacts of duration of ligation and administration for hyperuricemia, as well as the intervention time (time point) on the combined hyperuricemia and periodontitis model is difficult to predict.

### SUMMARY

To overcome the shortcomings of existing technology, the purpose of the present application is to provide a composite mouse model for simulating the exacerbation of periodontitis by hyperuricemia and a model for alleviating periodontitis exacerbated by hyperuricemia through uric acid-lowering treatment in mice. The established mouse model can not only increase the level of uric acid in a short time, but also induce periodontitis mildly and non-invasively. This patent effectively establishes a mouse model that simulates hyperuricemia-exacerbated periodontitis, successfully fills gaps in animal models related to hyperuricemia and periodontitis. It plays an important role in exploring the complex pathological mechanisms of association between hyperuricemia and periodontitis and screening drugs that address the exacerbation of periodontitis by hyperuricemia.

To achieve the purpose of the present application, the patent provides a method to establish a composite mouse model that simulates hyperuricemia and periodontitis, including:
Injecting potassium oxonate into the mouse peritoneum continuously for 7 days to induce hyperuricemia;
Using a microscopic needle holder to place a ligated silk thread around the maxillary second molar of the mouse to establish periodontitis model on the 7th day;
Intraperitoneally injecting potassium oxonate into the mouse for another 7 days (days 8 to 14) to establish a mouse model of accelerated periodontal disease in hyperuricemia.

In some embodiments of the present application, after intraperitoneally injecting potassium oxonate into the mouse continuously for another 7 days (days 8 to 14) to establish the composite mouse model, the method further includes: continuing to intraperitoneally inject potassium oxonate into the mouse for 14 days (days 15 to 28).

In some embodiments of the present application, after continuing to intraperitoneally inject potassium oxonate into the mouse for 14 days (days 15 to 28), the method further includes: on the 15th day, intraperitoneally injecting allopurinol continuously for 14 days (days 15 to 28) to decrease uric acid (days 15 to 28).

In some embodiments of the present application, the frequency of intraperitoneal injection of potassium oxonate is once a day, the injection dose is 600 mg/kg/d.

In some embodiments of the present application, the frequency of intraperitoneal injection of allopurinol is once a day, the injection dose is 5 mg/kg/d.

In some embodiments of the present application, intraperitoneally injecting potassium oxalate into the mouse includes: gently grasping the mouse and fixing the mouse in a palm, then intraperitoneally injecting potassium oxonate into the mouse, the concentration of potassium oxonate storage solution is 30 mg/mL.

In some embodiments of the present application, intraperitoneally injecting allopurinol into the mouse includes: gently grasping the mouse and fixing the mouse in a palm, then intraperitoneally injecting the allopurinol stock solution, the concentration of allopurinol stock solution is 0.5 mg/mL.

In some embodiments of the present application, using the microscopic needle holder to place ligated silk thread around the maxillary second molar of the mouse to establish periodontitis model on the 7th day includes:
Anesthetizing and fixing the mouse, exposing the maxillary molar area, cleaning the maxillary molar and gingiva, and drying thoroughly;
Using the surgical silk thread to fix the upper jaw of the mouse on a mouse plate, holding the surgical silk thread by the end of needle holder, then passing the surgical silk thread through an interdental space between the second molar and third molar;
Winding the surgical silk thread from distal buccal surface of the second molar to mesial buccal surface of the second molar, clamping the silk thread, then passing the silk thread through an interdental space between the first molar and second molar;
Tying the silk thread on the palatal side of the second molar;
Securing the silk thread firmly with three surgical knots, then cutting off excess silk thread with spring scissors.

In some embodiments of the present application, anesthetizing and fixing the mouse, exposing the maxillary molar area includes: anesthetizing the mouse intraperitoneally and fixing the mouse on a mouse board with abdomen facing; tilting the mouse board at a certain angle, a mouth opener is used to open the mouth so that researchers can see the maxillary molar area clearly, and researchers wear a head-mounted light to provide a view of the molar area;

Cleaning the maxillary molar and gingiva around the tooth and drying thoroughly includes: clean the maxillary molar and the gingiva with saline solution, and then wiping it with dry cotton balls.

In some embodiments of the present application, 5-0 surgical silk thread is ligated, microneedle holder is applied to hold needle, and the microneedle holder is W40350.

In some embodiments of the present application, the mouse is male C57BL/6 mouse aged 6 to 8 weeks.

To achieve the purpose of the present application, the present application further provides a hyperuricemia-exacerbated periodontitis mouse model and a model for relieving periodontal problems with uric acid-lowing treatment. These models are established as described above.

To achieve the purpose of the present application, the hyperuricemia-exacerbated periodontitis mouse model can be applied in screening drugs for treating periodontitis accompanied by hyperuricemia, wherein the composite mouse model is established as described above.

Compared with current technology, the present application can make sense in following aspects.

1. The present application effectively fills the gap in animal models related to hyperuricemia and periodontitis, innovatively simulates a composite mouse model of the exacerbation of periodontitis by hyperuricemia for the first time. It shows great application value in exploring the complex pathological mechanisms associated with the two diseases and screening drugs that suppress periodontal inflammation due to hyperuricemia. This mouse model helps to broaden the ideas and methods of research in this field to a certain extent.

2. The establishment of a hyperuricemia-exacerbated periodontitis mouse model in the present application only takes 14 days and making the mouse model stable only takes 28 days. Established in a short time, this mouse model is stable and reliable.

3. The present application induces periodontitis through silk thread ligation of the second maxillary molar of mice based on the successful establishment of a hyperuricemia model. In the time sequence, hyperuricemia is first and then periodontitis following. This can better explain the aggravating effect of hyperuricemia on periodontitis reasonably, and the modeling sequence is more scientific.

4. In the present application, the intraperitoneal injection of potassium oxonate at a dose of 600 mg/kg/d is used to induce and maintain the hyperuricemia model, which can not only keep the concentration of the uric acid stably at a higher level, but also avoid disadvantages of renal inflammatory reaction caused by long-term administration of potassium oxonate.

5. In the present application, allopurinol was injected intraperitoneally at a dose of 5 mg/kg/d to reduce blood uric acid levels, which further verified the exacerbation effect of hyperuricemia on periodontitis.

6. In the present application, the time points of the administration of potassium oxonate, allopurinol and ligation were reasonably selected. The model in the present application is in a complex state of coexistence of hyperuricemia and periodontitis from the 14th day. Afterward, the hyperuricemia model was maintained stably by continuous intraperitoneal injection of potassium oxonate, or on this basis, continuous intraperitoneal injection of allopurinol was performed to reduce blood uric acid levels from the 15th day. Based on the relevant research, the impact of hyperuricemia on different stages of periodontitis can be studied (relevant studies show that 0 to 14 days of ligation is the acute phase, and 14 to 21 days is the chronic phase).

### BRIEF DESCRIPTION OF THE DRAWINGS

FIG. 1A is a modeling flow chart according to an embodiment of the present application.

FIG. 1B shows the blood uric acid, creatinine, and urea nitrogen levels of each experimental group in the embodiment of the present application.

FIG. 1C shows the uric acid level of the silk thread in the true silk ligation group according to the embodiment of the present application.

FIG. 1D shows the blood xanthine/hypoxanthine levels and xanthine oxidase activity of each experimental group in the embodiment of the present application.

FIG. 1E shows the blood lipid and blood glucose levels of each experimental group in the embodiment of the present application.

FIG. 1F shows the body weight changes of each experimental group in the embodiment of the present application.

FIG. 1G shows the weight of each organ in each experimental group according to the embodiment of the present application.

FIG. 2A shows the three-dimensional reconstruction and parameter analysis results of the maxillary Micro-CT scan at the endpoint of the experiment in each experimental group according to the embodiment of the present application.

FIG. 2B shows the infiltrating leukocyte, TRAP⁺ cell, CD68⁺ cell and CD86⁺ cell number in periodontium of maxillary bones of mice at the experimental endpoint of each experimental group according to the embodiment of the present application.

FIG. 3A is an analysis of the mRNA expression levels of gingival inflammatory factors of mice at the experimental endpoint of each experimental group according to the embodiment of the present application.

FIG. 3B shows the mRNA sequencing analysis results of the gingival tissue of mice at the experimental endpoint of each experimental group according to the embodiment of the present application.

FIG. 4A is a differential analysis of the periodontal microbial community composition of mice in each experimental group according to the embodiment of the present application.

FIG. 4B is a correlation analysis of clinical factors and periodontal microbial components of mice in each experimental group according to the embodiment of the present application.

FIG. 5 is an analysis of the oxidative stress response levels in mice in each experimental group according to the embodiment of the present application.

FIG. 6 is an analysis of the expression levels of the NLRP3 inflammasome pathway in mice in each experimental group according to the embodiment of the present application.

### DETAILED DESCRIPTION OF THE EMBODIMENTS

The methods in the embodiments of the present application will be clearly and completely described below. The described embodiments are only some rather than all of the embodiments of the present application. Based on the embodiments of the present application, all other embodiments obtained by ordinary researchers in this field without creative work fall within the scope of the present application.

The establishment method for a composite mouse model that simulates the exacerbation of periodontitis by hyperuricemia in the embodiment of the present application is based on the establishment of a hyperuricemia model and a periodontitis model, creatively selecting the times of ligation as well the hyperuricemia administration induction, to combine the two and obtain a stable and reliable hyperuricemia-exacerbated periodontitis mouse model. Specifically, the establishment of the composite mouse model can include:
Step S1, establishing and maintaining the hyperuricemia model
Step S11, using a 1 mL syringe to draw a certain amount of pre-prepared potassium oxonate stock solution, and then weighing the injected mouse;
Step S12, gently grabbing the weighed mouse and securing the mouse in the palm, injecting the corresponding volume of potassium oxalate stock solution into the abdominal cavity of the mouse once daily via intraperitoneal injection, the injection dose of potassium oxalate is 600 mg/kg/day;
Step S13, repeating steps S11 and S12 with a fixed injection frequency every day, and continuously injecting potassium oxonate intraperitoneally for 14 days;
Step S14, continuously injecting potassium oxonate intraperitoneally for 14 days. At this time, the hyperuricemia model has been successfully established. To maintain a stable and high uric acid concentration, repeating steps S11 and S12 from the 15th day, and performing potassium oxonate intraperitoneal injection daily (the dose and frequency of daily injections remained unchanged) until the end of the experiment;
Step S2, establishing the periodontitis model;
Step S21, first anesthetizing and fixing the mouse, exposing the maxillary molar area, cleaning the maxillary molars and buccal-lingual gingiva with normal saline, and drying them thoroughly;
Step S22, fixing the upper jaw of the mouse on the mouse board by using surgical suture, clamping the surgical suture with the end of microneedle holder, and then passing the surgical suture through the interdental space between the second molar and the third molar;
Step S23, winding the surgical suture from the distal buccal of the second molar to the mesial buccal surface of the second molar, continue to hold the surgical suture in the same way as S22, and passing the surgical suture through the interdental space between the first molar and the second molar;
Step S24, tying the silk thread on the palatal side of the second molar by a needle holder (pay attention to removing the loose part);
Step S25, securing the silk thread firmly with three surgical knots, and cutting off the excess silk thread with spring scissors.

Step S3, establishing a hyperuricemia-periodontitis model;
Step S31, repeating steps S11 and S12, and continuously injecting potassium oxonate intraperitoneally for 7 days to establish the hyperuricemia model;
Step S32, repeating all operations in step S2 to establish a mouse periodontitis model;
Step S33, repeating steps S11 and S12, and continue to continuously inject potassium oxonate intraperitoneally for 21 days (days 8 to 28);
Step S34, on the 28th day, euthanizing the mouse 1 hour after the intraperitoneal injection of potassium oxonate, harvesting the samples to detect blood biochemistry, renal function, and renal pathology of the mouse, as well as the destruction and inflammation of the maxillary periodontal tissues.
Step S4, a model for alleviating the periodontitis aggravated by hyperuricemia in the mouse by uric acid-lowering treatment;
Step S41, repeating steps S11 and S12, and intraperitoneally injecting potassium oxonate continuously for 7 days to establish a hyperuricemia model;
Step S42, repeating all operations in step S2 to establish a periodontitis mouse model;
Step S43, repeating steps S11 and S12, and continue to intraperitoneally inject potassium oxonate continuously for 21 days (days 8 to 28);
Step S44, using a 1 mL syringe to draw a certain amount of pre-prepared allopurinol stock solution, and then weighing the injected mouse;
Step S45, gently grasping the weighed mouse and fixing it on the palm, according to the weight of the mouse, injecting corresponding volume of allopurinol stock solution into the abdominal cavity of the mouse once a day by intraperitoneal injection, the injection dose of allopurinol stock solution is 5 mg/kg/d;
Step S46, repeating steps S44 and S45 at a fixed time every day, and intraperitoneally injecting allopurinol continuously for 14 days (days 15 to 28).

The solution of the present application will be further described in detail below through specific examples and experimental results.

### Example 1

### 1. Materials

Fifty C57BL/6 mice, 18-22 g, 6-8 weeks old, were provided by the Guangdong Provincial Animal Experiment Center and raised in an SPF-level environment at the Animal Center of Guangdong Huawei Testing Co., Ltd. This experiment was approved by the Animal Ethics Committee of Guangdong Provincial Medical Experiment Animal Center. The 5-0 non-absorbable braided silk thread was provided by Johnson & Johnson, model Ethicon Mousse SA82G. Micro-CT was provided by Bruker Company of Germany, the model number was skyscan1172. Potassium oxonate (PO), sodium carboxymethylcellulose (CMC-Na), and allopurinol (Allo) were provided by Sigma Company, and their model numbers were 156124-25G, C5678-500G and A8003-25G respectively.

### 2. Reagent Preparation

Potassium oxonate stock solution (30 mg/mL): potassium oxonate (30 mg)+0.5% sodium carboxymethylcellulose (1 mL);

0.5% sodium carboxymethylcellulose: sodium carboxymethylcellulose powder (5 g)+normal saline (1 L);

Allopurinol (0.5 mg/mL): Allopurinol (60 mg)+NaOH (1 M, 3 mL)+normal saline (117 mL).

### 3. Experimental Grouping (Plus Another Group)

NuC group: normal control group, intraperitoneally injected with 0.5% CMC-Na, and the bilateral maxillary second molars were not ligated (n=10);

NuP group: experimental periodontitis group, intraperitoneally injected with 0.5% CMC-Na, and the bilateral maxillary second molars were ligated (n=10);

HuC group: hyperuricemia group, intraperitoneally injected with PO, and the bilateral maxillary second molars were not ligated (n=10);

HuP group: hyperuricemia combined with periodontitis group, intraperitoneally injected with PO, and bilateral maxillary second molars were ligated (n=10);

HuP+Allo group: hyperuricemia combined with periodontitis+urate-lowering treatment group, intraperitoneally injected with PO, the bilateral maxillary second molars were ligated and intraperitoneally injected with Allo (n=10).

4. Experimental methods and procedures

(1) After 50 male C57BL/6 mice arrived at the animal center of Huawei Testing Co., Ltd., the temperature: 20° C.-24° C., relative humidity: 50%-70%, light rhythm: 12 D: 12 L, working illumination: 150 lx˜300 lx, air flow and wind speed 0.1˜0.2 m/s, noise≤60 dB, adaptive feeding for 1 week.

(2) 50 mice were randomly divided into NuC, NuP, HuC, HuP, and HuP+Allo groups, with 10 mice in each group. Body weight was measured at the same time point once a day before and after the experiment, accurate to 0.01 g.

Mice in the NuC and NuP groups were intraperitoneally injected with 0.5% CMC-Na at a fixed frequency once a day (the injection volume was consistent with the PO volume required for mice under the same weight) for 28 consecutive days. The mice in the NuP group were ligated with 5-0 silk sutures on bilateral maxillary second molars by a microneedle holder on the 7th day. The mice in the HuC and HuP groups were intraperitoneally injected with PO at a fixed frequency once a day, the injection dose was 600 mg/kg/d (for example, for a mouse with a weight of 25 g, the volume of the corresponding potassium oxalate stock solution (30 mg/mL) to be injected in one day was 0.5 mL), and the injection lasted 28 days. On the 7th day, the mice in the HuP group were ligated with 5-0 silk sutures on bilateral maxillary second molars by a microneedle holder. On the 15th day, in the HuP+Allo group, Allo was intraperitoneally injected at a fixed frequency once a day based on the HuP group, the injection dose was 5 mg/kg/d (for example, for a mouse with the weight of 25 g, the volume of the corresponding potassium oxalate stock solution (0.5 mg/mL) to be injected in one day was 0.25 mL), and the injection lasted 14 days. The mice in the NuP, HuP, and HuP+Allo groups were checked for the presence of the ligation silk thread on days 1, 3, 7, 10, 14, and 21 after ligation. If the silk thread fell off, they needed to be re-ligated in time and recorded (all ligated mice in the present application kept the silk threads firmly in place during the ligation period, and no silk threads fell off). The specific method of ligation was as follows.

After the mice were intraperitoneally anesthetized with 1.5% sodium pentobarbital (dose: 40 mg/kg), the mice were fixed on the mouse board with abdomen facing, the mouse board was tilted at a certain angle and then fixed. A homemade mouth opener was used to stably open the mouth so that researchers can see the maxillary molar area clearly. A head-mounted light was used to provide a view of the molar area. The maxillary molars and buccal-lingual gingiva were cleaned with normal saline and dried thoroughly. Like dental floss, 5-0 surgical suture was inserted below the contact points of the maxillary first and second molar, and the second and third molar by the microscopic needle. Silk thread wrapped around the second molar. And then the needle holder was used to tie the suture on the palatal side of the second molar (pay attention to removing the loose part). The suture was firmly secured with three surgical knots, and the excess suture was cut off with spring scissors.

At the end of the experiment (1 hour after intraperitoneal injection of potassium oxonate/sodium carboxymethylcellulose on days 28), the mice were euthanized. The whole blood samples were collected and the plasmas were separated. Renal function, blood lipid levels, blood glucose levels and purine metabolism levels of mice were detected. The internal organs including kidneys, livers, spleens, hearts, lungs, and epididymal adipose tissue were collected. After weighing, some tissues were fixed in 4% paraformaldehyde, and the rest tissues were frozen in liquid nitrogen for half an hour and then stored in −80° C. refrigerator. After checking the silk threads in the ligation group, the silk threads ligated near the maxillary second molar were removed, and part of the silk threads were transferred to a 1.5 mL EP tube containing 100 μL PBS. The level of uric acid dissolved in PBS was detected. The rest silk threads were frozen in liquid nitrogen for half an hour and then stored in −80° C. refrigerator. The maxillary bones were separated and soaked in 4% paraformaldehyde solution for 24 to 48 hours. One side of the maxillary bones was used for scanning Micro-CT and H&E staining analysis. The other side was used to harvest gingiva, and the gingiva was frozen in liquid nitrogen for half an hour, stored in-80° C. refrigerator.

### 5. Experimental Results

1. The mouse model of hyperuricemia was successfully constructed. PO-induced hyperuricemia is characterized by increased blood uric acid and creatinine, but no changes in blood glucose and blood lipid levels.

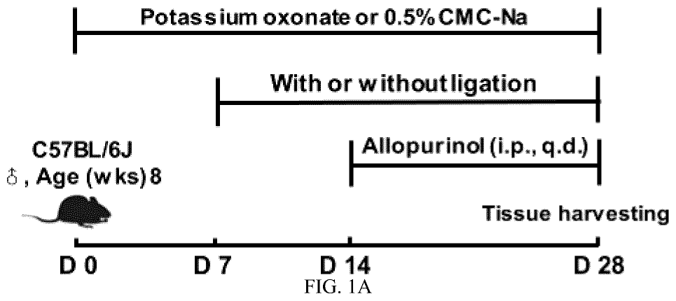

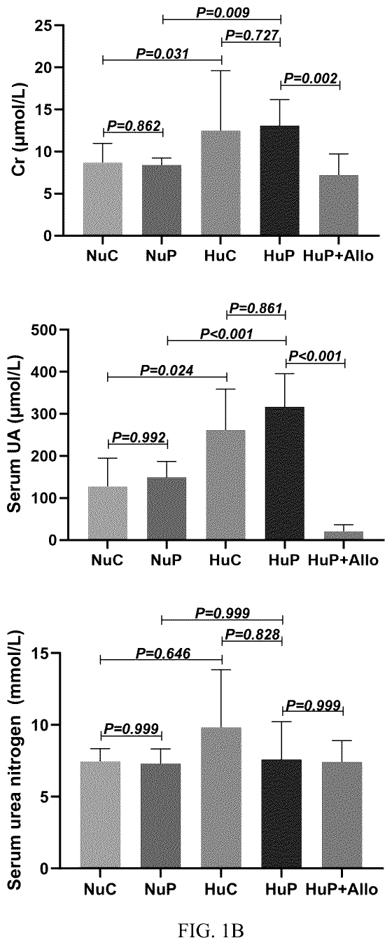

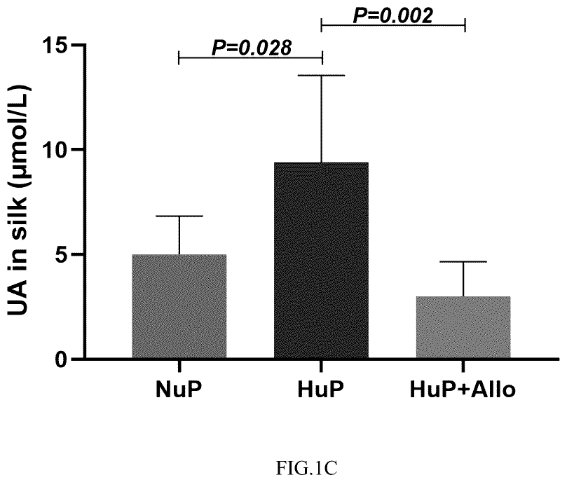

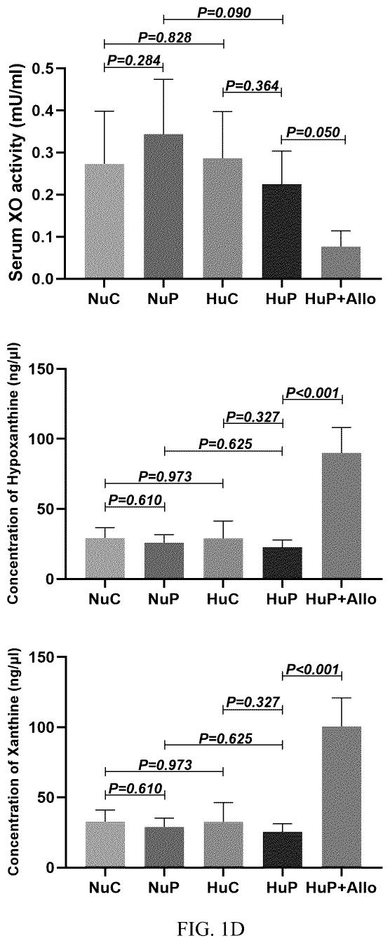

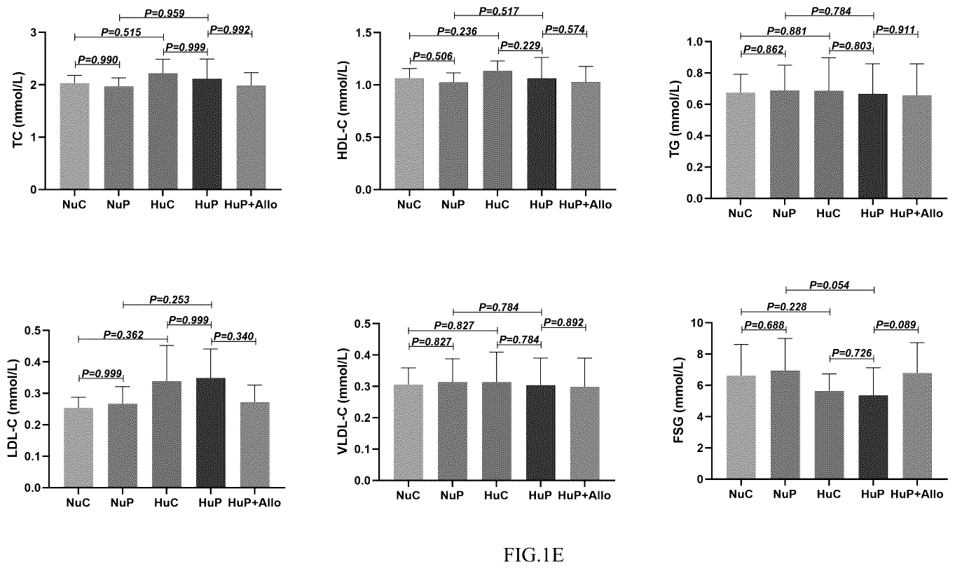

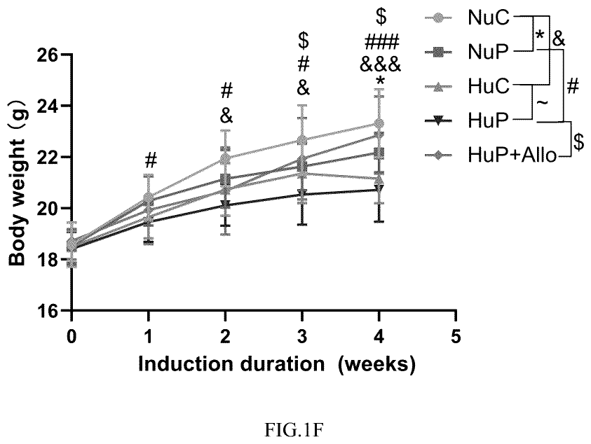

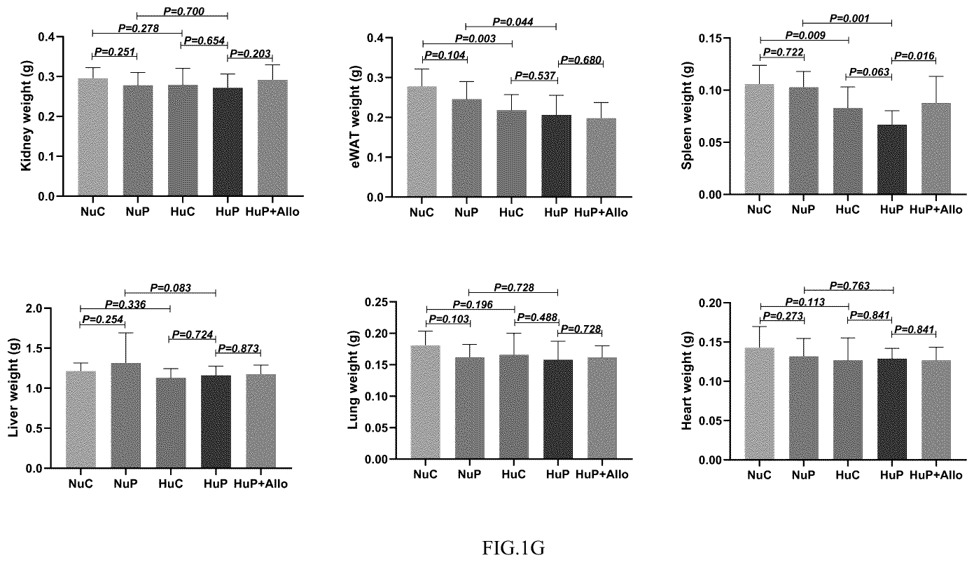

After 28 days of PO induction, compared with normal uric acid mice (Nu), regardless of whether periodontal ligation was performed, blood uric acid in hyperuricemia mice (Hu) increased by more than 2 times, and creatine increased by approximately 1.5 times than healthy groups. After allopurinol urate-lowering treatment (HuP+Allo vs. HuP), the blood uric acid levels and creatinine levels of mice were significantly reduced (P<0.001) (FIG. 1B). In addition, in the mice with periodontal ligation, PO (HuP vs. NuP) upregulated the uric acid in the silk threads, while allopurinol intervention (HuP+Allo vs. HuP) significantly reduced the uric acid (FIG. 1C). PO injection and ligation had no significant effect on activity of xanthine oxidoreductase and levels of its substrate hypoxanthine/xanthine upstream of UA in serum (FIG. 1D). Allopurinol lowered the activities of xanthine oxidoreductase while enhancing the hypoxanthine/xanthine level in serum (FIG. 1C, D).

PO injection and ligation had no significant effect on fasting blood lipids (TC, TG, HDL-C, LDL-C, VLDL-C) and blood glucose (P>0.05) (FIG. 1E). PO administration led to a reduction in body weight, with decrease observed from D14 (5.7%) to D28 (9.3%) in the controls without periodontitis (HuC vs. NuC) and from D7 (4.1%) to D28 (6.7%) in periodontitis mice (HuP vs. NuP). Additionally, periodontal ligation caused a 4.9% reduction in body weight at D28 in Nu mice (NuP vs. NuC). Interestingly, allopurinol treatment abrogated the PO-induced weight loss in ligated mice (FIG. 1F). PO and ligation had no significant effects on most systemic organs harvested. However, epididymal white adipose tissue weight decreased with both PO and ligation, and spleen weight decreased with PO (FIG. 1G).

2. The presence of hyperuricemia exacerbated the alveolar bone destruction and inflammatory response in the periodontal tissues of mice with periodontitis.

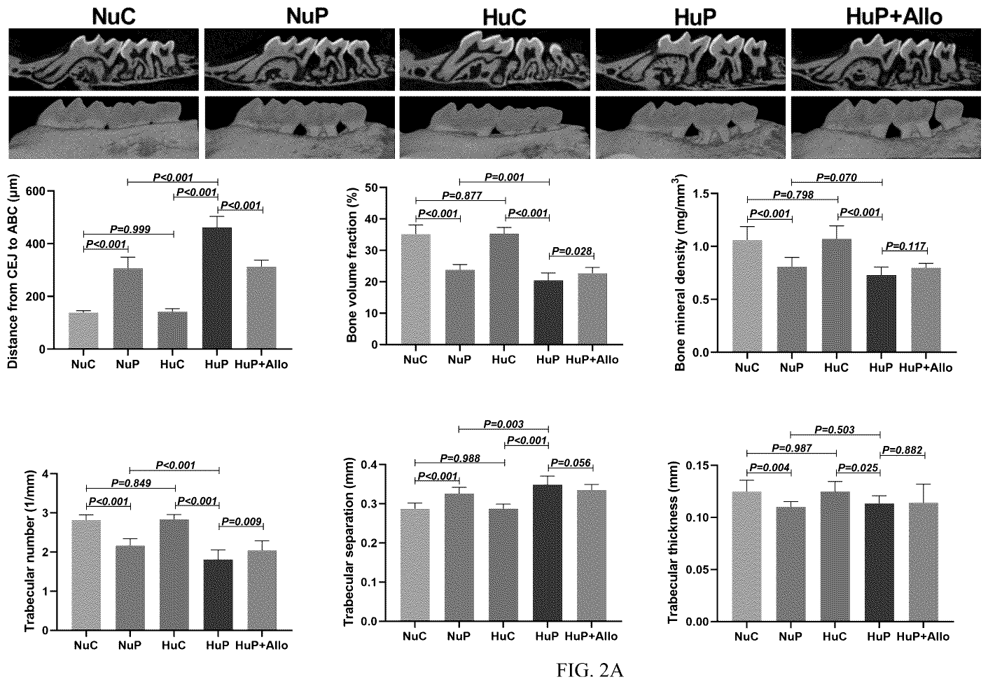

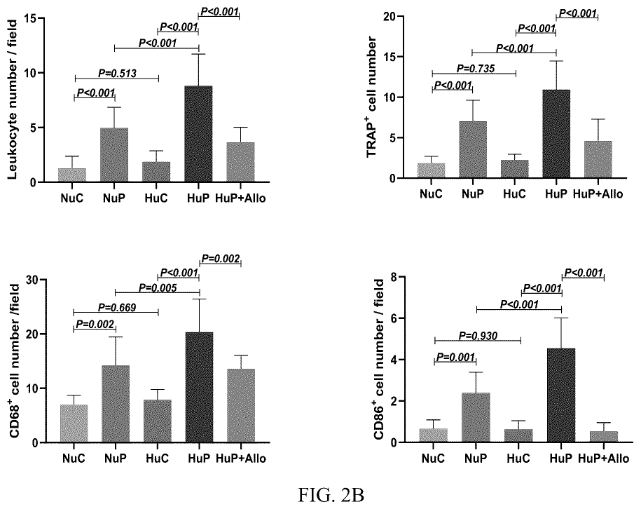

Micro-CT analysis showed that compared to the periodontal healthy group, the periodontal ligation group showed a significant increase in the average distance from the cement-enamel junction to the alveolar bone crest (CEJ-ABC) and the trabecular spacing (Tb.Sp) of the maxillary second molar (P<0.001), and bone mineral density (BMD), bone volume fraction (BVF=BV/TV), trabecular bone number (Tb.N) and trabecular bone thickness (Tb.Th) were significantly decreased (P<0.001) (FIG. 2A). H&E and TRAP staining showed that the number of inflammatory cells and osteoclasts increased in the periodontal tissues of the ligation group (FIG. 2B). These results confirmed that the mouse experimental periodontitis model was successfully established. Hyperuricemia exacerbates alveolar bone destruction-related indicators (BVF, Tb.N, Tb.Th) in mice with periodontitis (HuP vs. NuP), and also had a tendency to aggravate BMD (P=0.070) (FIG. 2A). Hyperuricemia increases the number of inflammatory cells and osteoclasts in periodontal tissues of mice with periodontitis; in contrast, allopurinol ameliorated the periodontal condition of HuP mice, including alveolar bone resorption, the number of inflammatory cells and osteoclasts (FIG. 2A, FIG. 2B).

3. Hyperuricemia interfered with periodontal immune response.

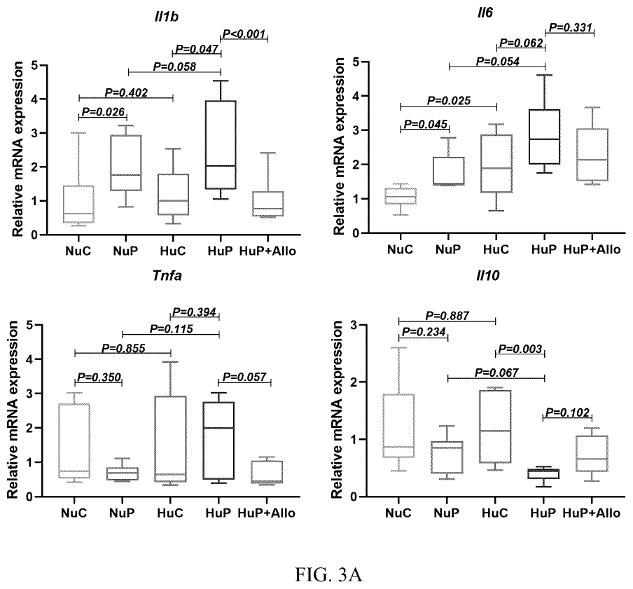

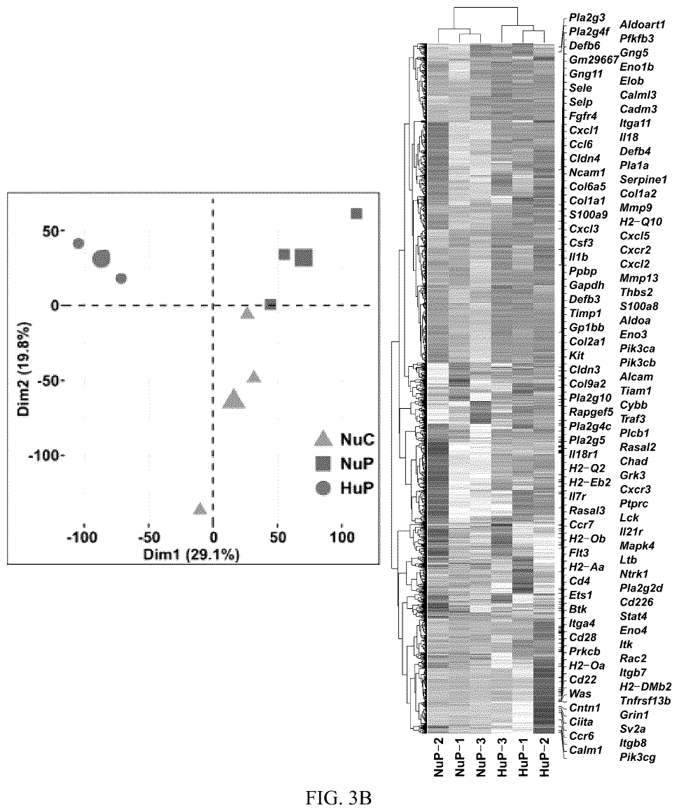

Immunohistochemistry results showed that the number of macrophages (CD68+), M1 macrophages (CD86+) and osteoclasts in the periodontal tissues of ligated mice (HuP and NuP) significantly increased due to hyperuricemia (FIG. 2B). RT-qPCR found that hyperuricemia upregulated the mRNA levels of IIIb (P=0.058) and II6 (P=0.054) in the gingiva of mice with periodontal ligation (FIG. 3A). Allo reduced the total number of macrophages and the number of M1 macrophages, and tended to downregulate the mRNA levels of IIIb (P<0.001) and Tnfa (P=0.057) (FIG. 2B, FIG. 3A). Subsequent mRNA sequencing further revealed the periodontal tissues response in hyperuricemia. Among the 16,746 identified genes, 2105 genes (640 up-regulated and 1465 down-regulated) were differentially expressed between the NuP and HuP groups (FIG. 3B). KEGG enrichment analysis found 17 pathways related to hyperuricemia (excluding disease-related pathways) (HuP vs. NuP, P<0.05), most of which were related to immune response (FIG. 3B).

4. Hyperuricemia aggravated periodontal microbiome imbalance.

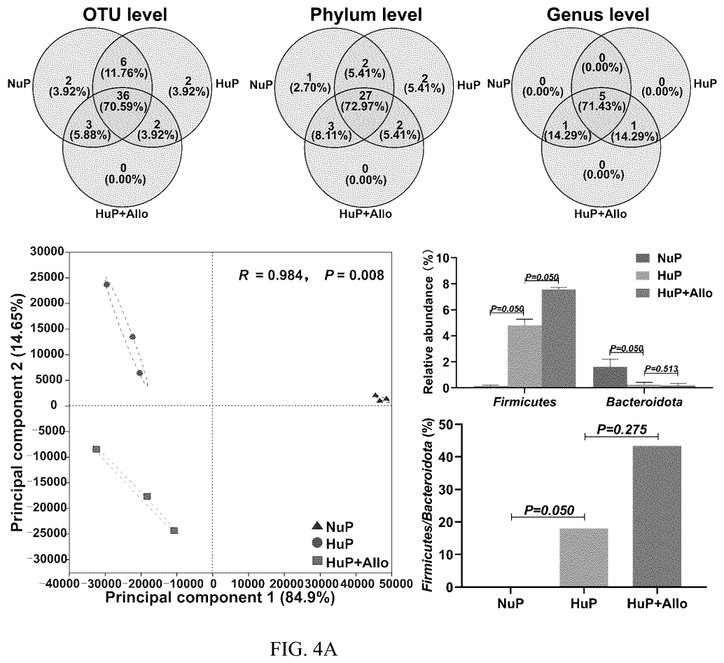

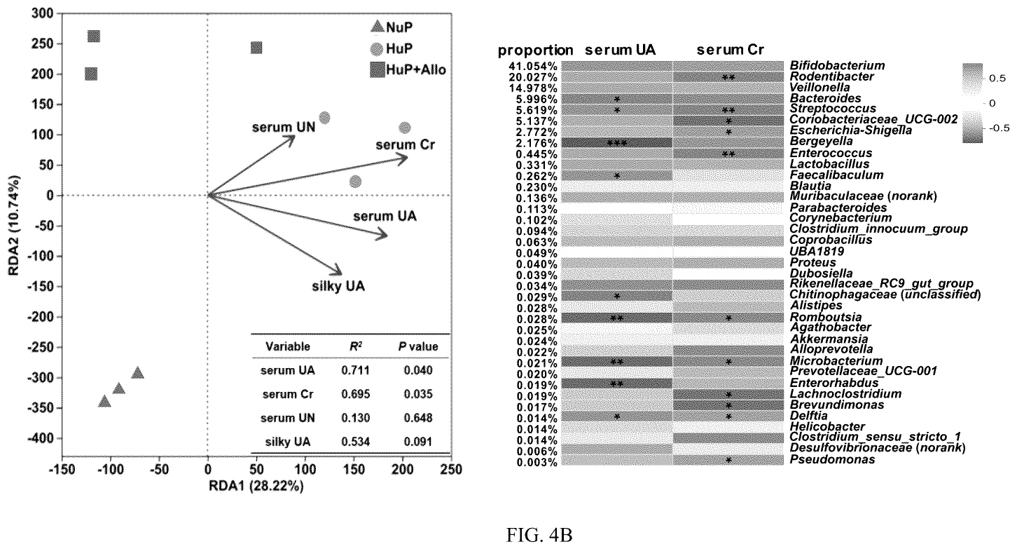

Microbiome of periodontal ligation threads analyzed by 16S rRNA sequencing. The periodontal microbiota of the three ligation groups (NuP, HuP, HuP+Allo) contained 51 OUT, 7 phyla, and 37 genera. A majority (˜70%) of the microbiota were shared among the three groups. Principal component analysis showed a distinct separation of microbiome compositions at the phylum level among the three groups. Hyperuricemia increased the abundance of Firmicutes and Proteobacteria but decreased the abundance of Bacteroidota and Actinobacteria in ligated mice; the abundance of Firmicutes was further upregulated in the urate-lowering treatment group (HuP+Allo) (FIG. 4A).

Among 12 clinical factors (including organ weight, serum cytokines, blood glucose, blood lipids, and renal function parameters), only serum uric acid and creatinine levels were associated with microbial composition. The related heat map showed that the abundance of multiple bacterial genera was significantly correlated with the contents of serum uric acid and creatinine (FIG. 4B).

5. Excessive uric acid aggravated the oxidative stress response of periodontal tissues.

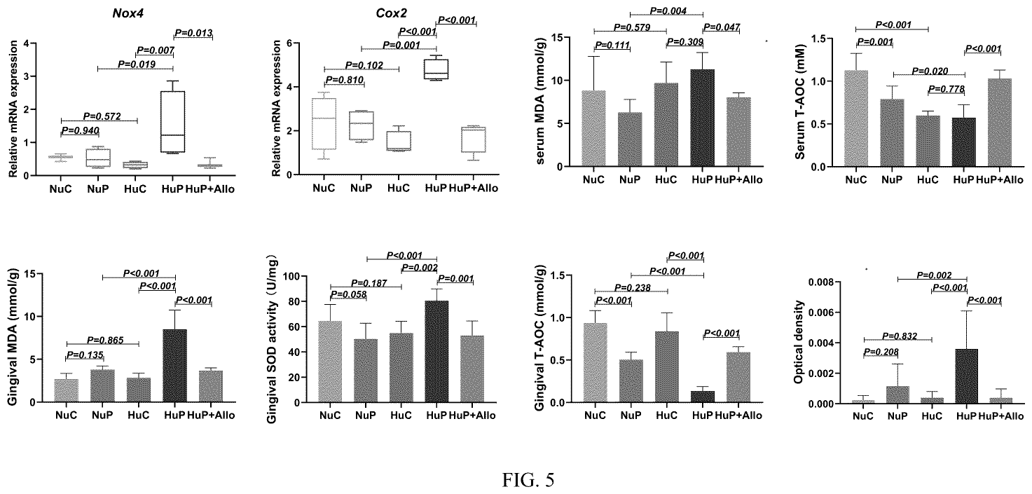

Compared with the periodontal healthy control group (NuC), periodontitis group (NuP) significantly reduced the total antioxidant capacity (T-AOC) of gingiva and showed a tendency to downregulate SOD activity (P=0.058). Periodontitis did not affect the concentration of the lipid peroxidation product MDA and the mRNA levels of the prooxidative genes Cox2 and Nox4. Hyperuricemia further reduced T-AOC in periodontitis groups (HuP and NuP). Hyperuricemia also enhanced SOD activity, increased MDA content, and the expression of Cox2 and Nox4 at the mRNA level. Immunohistochemistry confirmed that similar changes in NOX4 occurred in hyperuricemia-induced periodontal sections (HuP and NuP). Allopurinol reversed hyperuricemia-induced changes in these oxidative stress-related parameters (HuP+Allo vs. HuP) (FIG. 5).

6. Excessive UA activated the NLRP3 inflammasome pathway in periodontitis.

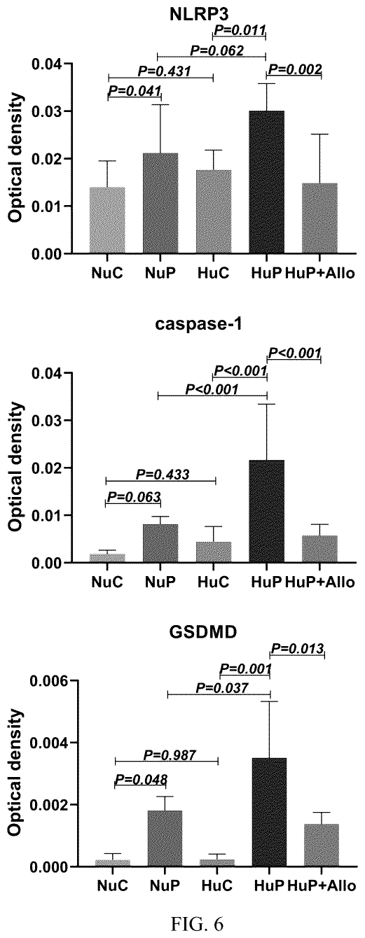

The NLRP3 inflammasome pathway was activated in periodontal tissues, which is manifested by a significant increase in the expression of NLRP3 and an upward trend in the expression of caspase-1 at the protein level. Hyperuricemia increased caspase-1 protein expression, and NLRP3 levels also tended to increase in periodontitis patients. In addition, the protein level of gasdermin D, a thermal protein deposition-related factor downstream of caspase-1, was also increased by periodontitis, and hyperuricemia further increased the caspase-1 protein level in ligated mice. Allopurinol could inhibit the increase of NLRP3, caspase-1 and gasdermin D caused by hyperuricemia (FIG. 6).

Therefore, the establishment method of the hyperuricemia-exacerbated periodontitis mouse model provided by the present application includes: intraperitoneally injecting potassium oxonate (PO) at a fixed frequency once a day for 7 days to establish a hyperuricemia model; on the 7th day, ligating the maxillary second molar of the mouse to establish a periodontitis model; continuing to intraperitoneally inject potassium oxonate (PO) at a fixed frequency once a day on the days 8 to 14, to establish a mouse model of hyperuricemia combined with periodontitis; thereafter, injecting potassium oxonate at a fixed frequency once a day for 14 consecutive days (days 15 to 28) to maintain the hyperuricemia model and construct a stable hyperuricemia combined with periodontitis mouse model; on the 15th day, simultaneously injecting allopurinol intraperitoneally for 14 consecutive days to decrease uric acid, and establishing a model for alleviating periodontitis exacerbated by hyperuricemia through uric acid-lowering treatment. In the present application, the hyperuricemia model constructed using potassium oxonate (PO) can not only increase uric acid levels in a short period of time, but also maintain stable uric acid levels for a longer period through continuous administration during the maintenance period. Combined with the construction of a mild and non-invasive periodontitis model, a mouse model that simulates the exacerbation of periodontitis by hyperuricemia and a model for alleviating periodontitis exacerbated by hyperuricemia through uric acid-lowering treatment were innovatively proposed. The models established in the present application not only observed the aggravation of alveolar bone resorption and periodontal tissues inflammation by hyperuricemia, but also observed the aggravation of periodontal microbial dysbiosis, oxidative stress and key signaling pathways by hyperuricemia. These data support the aggravating effect of hyperuricemia on periodontitis from multiple dimensions such as morphological/histological/molecular levels and comprehensively describe the important characteristics of this composite model. This effectively fills gaps in animal models related to hyperuricemia and periodontitis, shows great application value in exploring the complex pathological mechanisms related to the two diseases. This mouse model helps to broaden the ideas and methods of research in this field to a certain extent.

So far, researchers realized that although the embodiments of the present application have been described in detail herein, without departing from the spirit and scope of the present application, many other variations or modifications consistent with the principles of the present application can still be directly determined or deduced according to the content disclosed in the present application. Accordingly, the scope of the present application should be understood and deemed to cover all such other variations or modifications.

What is claimed is:

1. An establishment method of a composite mouse model for simulating exacerbation periodontitis by hyperuricemia, comprising:
Continuously intraperitoneally injecting potassium oxonate into a mouse for 7 days to establish hyperuricemia;
Using a microscopic needle holder to ligate the maxillary second molar of the mouse by a surgical silk thread to establish a periodontitis model on the 7th day; and
Continuously intraperitoneally injecting potassium oxonate into the mouse for another 7 days (days 8 to 14) to establish a composite mouse model for simulating the exacerbation of periodontitis by hyperuricemia.

2. The establishment method of claim 1, wherein after continuing to continuously intraperitoneally injecting potassium oxonate into the mouse for another 7 days (days 8 to 14) to establish the composite mouse model for simulating the exacerbation of periodontitis by hyperuricemia, the method further comprises: continuing to continuously intraperitoneally injecting potassium oxonate into the mouse for 14 days (days 15 to 28).

3. The establishment method of claim 2, wherein after continuing to continuously intraperitoneally injecting potassium oxonate into the mouse for 14 days (days 15 to 28), the method further comprises: on the 15th day, continuously intraperitoneally injecting allopurinol for 14 days for uric acid lowering treatment (days 15 to 28).

4. The establishment method of claim 2, wherein a frequency of intraperitoneally injecting potassium oxalate into the mouse is once a day, and the injection dose is 600 mg/kg/d.

5. The establishment method of claim 3, wherein a frequency of intraperitoneally injecting allopurinol into the mouse is once a day, and the injection dose is 5 mg/kg/d.

6. The establishment method of claim 2, wherein intraperitoneally injecting potassium oxalate into the mouse comprises: gently grasping the mouse and fixing the mouse in a palm, and intraperitoneally injecting potassium oxonate stock solution into the mouse, and wherein a concentration of the potassium oxonate stock solution is 30 mg/mL.

7. The establishment method of claim 3, wherein intraperitoneally injecting allopurinol into the mouse comprises: gently grabbing the mouse and fixing the mouse in a palm, and intraperitoneally injecting the allopurinol stock solution into the mouse, and wherein a concentration of allopurinol stock solution is 0.5 mg/mL.

8. The establishment method of claim 1, wherein using the microscopic needle holder to ligate the maxillary second molar of the mouse by a surgical silk thread to establish a periodontitis model on the 7th day comprises:
Anesthetizing and fixing the mouse, exposing the maxillary molar area, cleaning the maxillary molar and buccal-lingual gingiva, and drying thoroughly;
Using the surgical silk thread to fix the upper jaw of the mouse on a mouse plate, using an end of the needle holder to hold the surgical silk thread, and passing the surgical silk thread through an interdental space between a second molar and a third molar;
Winding the surgical silk thread from a distal buccal surface of the second molar to a mesial buccal surface of the second molar, clamping the surgical silk thread, and passing the surgical silk thread through an interdental space between a first molar and the second molar;
Using the needle holder to tie the surgical silk thread on a palatal side of the second molar; and
Securing the silk thread firmly with three surgical knots, and then cutting off excess silk thread with spring scissors.

9. The establishment method of claim 8, wherein anesthetizing and fixing the mouse, exposing the maxillary molar area comprises: anesthetizing the mouse intraperitoneally, and fixing the mouse on a mouse board with abdomen facing; tilting the mouse board at a certain angle and then fixing the mouse board, using a mouth opener to stably expose the maxillary molar area, and using a head-mounted light to provide a view of the molar area; and/or
Cleaning the maxillary molar and the buccal-lingual gingiva and drying thoroughly comprises: using saline to clean the maxillary molar and the buccal-lingual gingiva, and then wiping it with dry cotton balls.

10. The establishment method of claim 8, wherein the thread is a 5-0 surgical silk thread, the needle holder is a microneedle holder, and the model number of the microneedle holder is W40350.

11. The establishment method of claim 1, wherein the mouse is a male C57BL/6 mouse aged 6 to 8 weeks.

12. A composite mouse model for simulating exacerbation of periodontitis and a model for improving periodontitis exacerbated by hyperuricemia using uric acid-lowering treatment in a mouse, wherein the model is obtained by the establishment method of claim 1.

13. Use of a composite mouse model for simulating exacerbation of periodontitis by hyperuricemia in screening a drug for treating periodontitis accompanied by hyperuricemia, wherein the composite mouse model is obtained by the establishment method of claim 1.
